(19) United States
(12) Patent Application Publication
(10) Pub. No.: US 2021/0290634 A1
(43) Pub. Date: Sep. 23, 2021

(54) METHODS AND COMPOSITIONS FOR REDUCING TOLERANCE TO OPIOID ANALGESICS USING IBOGAINE AND DERIVATIVES THEREOF

(71) Applicant: DEMERX, INC., Miami, FL (US)

(72) Inventors: Emeline MAILLET, Miami Beach, FL (US); Lawrence FRIEDHOFF, River Vale, NJ (US)

(21) Appl. No.: 17/339,357

(22) Filed: Jun. 4, 2021

Related U.S. Application Data
(63) Continuation of application No. 14/635,871, filed on Mar. 2, 2015.
(60) Provisional application No. 61/952,743, filed on Mar. 13, 2014.

(51) Int. Cl. A61K 31/55

(52) U.S. Cl. CPC A61K 31/55

(57) ABSTRACT

A method for modulating tolerance to an opioid analgesic in a patient undergoing opioid analgesic therapy, the method comprising interrupting or administering concurrently with said opioid analgesic therapy an amount of ibogaine, ibogaine derivative, or pharmaceutically acceptable salt and/or solvate thereof.

### CROSS-REFERENCE TO RELATED APPLICATIONS

This application claims benefit from U.S. Provisional Application No. 61/952,743, filed Mar. 13, 2014, which is hereby incorporated by reference in its entirety.

### FIELD OF THE INVENTION

This invention is directed to methods for reducing tolerance to opioids in a patient undergoing opioid analgesic treatment for pain comprising treating the patient with ibogaine, an ibogaine derivative, or a pharmaceutically acceptable salt and/or solvate thereof (hereinafter referred to as “ibogaine”) at a therapeutic dosage.

### STATE OF THE ART

Addictive opioid analgesic agents such as morphine are well-known and exceptionally potent analgesics. Such opioids operate as mu receptor agonists. Upon administration, opioids initiate a cascade of biological events including increased serotonin and dopamine expression. As is well known, continued use of many such opioids (especially at high doses) carries a significant risk of dependency/addiction. Indeed, potential addiction to such opioids is a serious issue that limits the therapeutic use of addictive opioids as analgesic agents. For example, the use of morphine as an analgesic is common among end stage patients suffering from serious pain where addiction is no longer a concern.

Drug tolerance to opioid analgesics is common, and may be psychological and/or physiological. A patient who has developed tolerance to the opioid analgesic is not necessarily addicted to or misusing the analgesic. Drug tolerance occurs when the patient's reaction to the drug is reduced, requiring an increase in dose to achieve the same desired effect. There are several potential methods for how tolerance develops, including receptor desensitization, receptor phosphorylation, receptor internalization or down-regulation, and up-regulation of inhibitory pathways.

Drug tolerance requires that the dosage of analgesic be increased in order to provide sustained analgesic effect. However, high doses of opioids may lead to serious complications and side effects, including physical dependence, addiction, respiratory depression, nausea, sedation, euphoria or dysphoria, decreased gastrointestinal motility, and itching.

It would be beneficial to provide a method for modulating opioid analgesic tolerance in a patient taking one or more opioid analgesics for the treatment of pain.

### SUMMARY

This invention is directed, in part, to the use of ibogaine to modulate tolerance to addictive opioid analgesic agents in a patient who has developed or is at risk of developing a tolerance for the analgesic. In such methods, effective analgesia can be achieved in a patient while resensitizing the patient to the addictive opioid analgesic. The term “resensitizing the patient” is used herein to refer to reducing, relieving, attenuating, and/or reversing tolerance to the analgesic. In one aspect, the resensitized patient obtains therapeutic effect from a lower dose of the opioid analgesic than before resensitization. In one aspect, the resensitized patient obtains improved therapeutic effect from the same dose of the opioid analgesic compared to before resensitization.

The use of ibogaine for the modulation of tolerance to opioid analgesic agents is limited due to potentially adverse side effects. For example, ibogaine exhibits undesirable stimulant and hallucinogenic properties, and in addition, can induce tremors. At conventional doses, ibogaine causes adverse side effects in a majority of patients receiving treatment. Thus, the use of ibogaine to modulate opioid tolerance is generally not favored due to the adverse side effects that can result from receiving a therapeutic dose according to conventional known methods.

It has been discovered that the use of ibogaine, ibogaine derivative, or pharmaceutically acceptable salt and/or solvate thereof imparts a dose dependent prolongation of the treated patient's QT interval, rendering higher dosing of ibogaine unacceptable. A prolonged QT interval is a marker of potential Torsades de Pointes, a serious arrhythmia that can result in death. For reasons that are not apparent, this prolongation increases in opioid addicted patients as compared to healthy individuals. Heretofore, it was unclear whether a therapeutic dose of ibogaine could be found that resulted in QT interval prolongation within an acceptable range. It is expected that other compounds that share ibogaine's core structure will have a similar prolongation effect on QT interval. See, U.S. Provisional Patent Application No. 61/945,746 filed Feb. 27, 2014 entitled METHOD FOR ACUTE AND LONG-TERM TREATMENT OF DRUG ADDICTION, which application is incorporated by reference in its entirety.

The current invention is predicated on the surprising discovery that treatment with a narrow dosage range of ibogaine, ibogaine derivative, or pharmaceutically acceptable salt and/or solvate thereof, between greater than about 1 mg/kg body weight and about 8 mg/kg body weight, provides a therapeutic modulation of in tolerance to opioid analgesics. Preferably, the dose range that provide both therapeutic results and an acceptable QT interval prolongation of less than 50 milliseconds is between about 1.3 mg per kg body weight and no more than about 4 mg per kg body weight and, more preferably between about 1.3 mg per kg body weight and no more than about 3 mg per kg body weight, or any subrange or subvalue within the aforementioned ranges.

In a preferred embodiment, the narrow therapeutic doses of ibogaine, ibogaine derivative, or pharmaceutically acceptable salt and/or solvate described above do not prolong the QT interval to unacceptable levels in human patients. In some embodiments, patients will be administered therapeutic doses of ibogaine, ibogaine derivative, or pharmaceutically acceptable salt and/or solvate thereof in a clinical setting with cardiac monitoring. In some embodiments, the patient will be pre-screened to evaluate tolerance for prolongation of QT interval, e.g., to determine whether the patient has any pre-existing cardiac conditions which would disqualify them from treatment with ibogaine.

In one embodiment, ibogaine, ibogaine derivative, or pharmaceutically acceptable salt and/or solvate thereof is administered concurrently with the opioid analgesic. In one embodiment, ibogaine, ibogaine derivative, or pharmaceutically acceptable salt and/or solvate thereof is administered after administration of the analgesic, for example one, two, three, four, eight, ten, twelve, 24 hours or more after administration of the analgesic. In one embodiment, one dose of ibogaine, ibogaine derivative, or pharmaceutically acceptable salt and/or solvate thereof is administered. In one embodiment, two or more doses of ibogaine, ibogaine derivative, or pharmaceutically acceptable salt and/or solvate thereof are administered. In one embodiment, the opioid analgesic is interrupted for a period of time while ibogaine, ibogaine derivative, or pharmaceutically acceptable salt and/or solvate thereof is administered. In one embodiment, a non-opioid analgesic is administered while the opioid analgesic is interrupted. In one embodiment, ibogaine, ibogaine derivative, or pharmaceutically acceptable salt and/or solvate thereof acts as an analgesic. In one embodiment, the opioid analgesic is not interrupted during ibogaine treatment.

In some embodiments, the therapeutic dose of ibogaine, ibogaine derivative, or pharmaceutically acceptable salt and/or solvate thereof administered to the patient is sufficient to provide an average serum concentration of about 50 ng/mL to about 850 ng/mL (area under the curve/24 hours), or any subrange or subvalue there between. In a preferred embodiment, the dose of ibogaine or pharmaceutically acceptable salt and/or solvate thereof administered to the patient provides an average serum concentration of about 50 ng/mL to about 400 ng/mL.

In some embodiments, the dose of ibogaine, ibogaine derivative, or pharmaceutically acceptable salt and/or solvate thereof that provides an average serum concentration of about 50 ng/mL to about 850 ng/mL is administered as a single dose. In some embodiments, the dose of ibogaine, ibogaine derivative, or pharmaceutically acceptable salt and/or solvate thereof that provides an average serum concentration of about 50 ng/mL to about 850 ng/mL is administered as multiple doses. In some embodiments, the aggregate dose of ibogaine, ibogaine derivative, or pharmaceutically acceptable salt and/or solvate thereof is from greater than about 1 mg/kg to about 8 mg/kg. In a preferred embodiment, the aggregate dose of ibogaine, ibogaine derivative, or pharmaceutically acceptable salt and/or solvate thereof is from greater than about 1 mg/kg to about 4 mg/kg. In another preferred embodiment, the aggregate dose of ibogaine, ibogaine derivative, or pharmaceutically acceptable salt and/or solvate thereof is from greater than about 1 mg/kg to 3 mg/kg.

In some embodiments, the serum concentration is sufficient to modulate said tolerance while maintaining a QT interval of less than 500 milliseconds (ms) during said treatment. In some embodiments, the therapeutic dose of ibogaine, ibogaine derivative, or pharmaceutically acceptable salt and/or solvate thereof provides prolongation of the QT interval of less than 80 ms. In one embodiment, the dose of ibogaine, ibogaine derivative, or pharmaceutically acceptable salt and/or solvate thereof provides prolongation of the QT interval of less than 50 ms. In some embodiments, the dose of ibogaine, ibogaine derivative, or pharmaceutically acceptable salt and/or solvate thereof provides prolongation of the QT interval of less than 30 ms. In a preferred embodiment, the dose of ibogaine, ibogaine derivative, or pharmaceutically acceptable salt and/or solvate thereof provides prolongation of the QT interval of less than 20 ms. In one embodiment, the patient is tested to determine QT interval and/or risk of prolongation before treatment with ibogaine, and if clinician determines that the QT prolongation would be unacceptable risk, ibogaine therapy will be contraindicated.

### DETAILED DESCRIPTION

It is to be understood that this invention is not limited to particular embodiments described, as such may, of course, vary. It is also to be understood that the terminology used herein is for the purpose of describing particular embodiments only, and is not intended to be limiting, since the scope of this invention will be limited only by the appended claims.

The detailed description of the invention is divided into various sections only for the reader's convenience and disclosure found in any section may be combined with that in another section. Unless defined otherwise, all technical and scientific terms used herein have the same meaning as commonly understood by one of ordinary skill in the art to which this invention belongs.

It must be noted that as used herein and in the appended claims, the singular forms “a”, “an”, and “the” include plural referents unless the context clearly dictates otherwise. Thus, for example, reference to “a compound” includes a plurality of compounds.

### I. Definitions

Unless defined otherwise, all technical and scientific terms used herein have the same meaning as commonly understood by one of ordinary skill in the art to which this invention belongs. As used herein the following terms have the following meanings.

The term “about” when used before a numerical designation, e.g., temperature, time, amount, concentration, and such other, including a range, indicates approximations which may vary by (+) or (−) 10%, 5% or 1%.

“Administration” refers to introducing an agent into a patient. Typically, an effective amount is administered, which amount can be determined by the treating physician or the like. Any route of administration, such as oral, topical, subcutaneous, peritoneal, intra-arterial, inhalation, vaginal, rectal, nasal, introduction into the cerebrospinal fluid, or instillation into body compartments can be used. The agent may be administered by direct blood stream delivery, e.g. sublingual, intranasal, or intrapulmonary administration.

The related terms and phrases “administering” and “administration of”, when used in connection with a compound or pharmaceutical composition (and grammatical equivalents) refer both to direct administration, which may be administration to a patient by a medical professional or by self-administration by the patient, and/or to indirect administration, which may be the act of prescribing a drug. For example, a physician who instructs a patient to self-administer a drug and/or provides a patient with a prescription for a drug is administering the drug to the patient.

“Periodic administration” or “periodically administering” refers to multiple treatments that occur on a daily, weekly, or monthly basis. Periodic administration may also refer to administration of ibogaine or salt and/or solvate thereof one, two, three, or more times per day. Administration may be via transdermal patch, gum, lozenge, sublingual tablet, intranasal, intrapulmonary, oral administration, or other administration.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

As used herein, [symbol] is a single bond or a double bond.

As used herein, the term “alkyl” refers to monovalent saturated aliphatic hydrocarbyl groups having from 1 to 12 carbon atoms, 1 to 10 carbon atoms, preferably 1 to 6 carbon atoms, and more preferably 1 to 3 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—). The term “Cₓ alkyl” refers to an alkyl group having x carbon atoms, wherein x is an integer, for example, C₃ refers to an alkyl group having 3 carbon atoms.

“Substituted alkyl” refers to an alkyl group having from 1 to 5, preferably 1 to 3, or more preferably 1 to 2 substituents selected from the group consisting of alkoxy, R²⁰—C(O)—, —NR²⁰C(O)R²⁰,

R²⁰—C(O)O—, —NR²⁰R²⁰, —C(O)NR²⁰R²⁰, —C(S)NR²⁰R²⁰, —NR²⁰C(O)NR²⁰R²⁰, —NR²⁰C(S)NR²OR²⁰, —O—C(O)NR²⁰R²⁰,
S(O)₂NR²⁰R²⁰, —O—S(O)₂NR²⁰R²⁰, —NR²⁰—S(O)₂NR²⁰R²⁰, —C(═NR²⁰)NR²⁰R²⁰, aryl, aryloxy, arylthio, azido, carboxyl, —C(O)O—R²¹, —NR²⁰—C(O)O—R²¹, —O—C(O)O—R²¹, cyano, cycloalkyl, cycloalkyloxy, cycloalkylthio, —NR²⁰C(═NR²⁰)N(R²⁰)₂, halo, hydroxy, hydroxyamino, alkoxyamino, —NR²⁰NR²⁰R²⁰, heteroaryl, heteroaryloxy, heteroarylthio, heterocyclic, heterocyclyloxy, heterocyclylthio, nitro, spirocycloalkyl,
SO₃H, —OS(O)₂—R²¹, —S(O)₂—R²¹, —C(S)—R²¹, thiocyanate, thiol, and alkylthio; each R²⁰ is independently selected from the group consisting of hydrogen, alkyl, cycloalkyl, aryl, heteroaryl, and heterocycle, or two R²⁰ groups attached to a common atom are optionally joined together with the atom bound thereto to form a heterocycle; and each R²¹ is independently selected from the group consisting of alkyl, cycloalkyl, aryl, heteroaryl, and heterocycle.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Preferred aryl groups include phenyl and naphthyl.

“Substituted aryl” refers to aryl groups which are substituted with 1 to 5, preferably 1 to 3, or more preferably 1 to 2 substituents selected from the group consisting of alkyl, substituted alkyl, alkoxy, —C(O)—R²⁰, —NR²⁰C(O)R²⁰,

R²⁰—C(O)O—, —NR²⁰R²⁰, —C(O)NR²⁰R²⁰, —C(S)NR²⁰R²⁰, —NR²⁰C(O)NR²⁰R²⁰, —NR²⁰C(S)NR²⁰R²⁰, —O—C(O)NR²⁰R²⁰,
S(O)₂NR²⁰R²⁰, —O—S(O)₂NR²⁰R²⁰, —NR²⁰—S(O)₂NR²⁰R²⁰, —C(═NR²⁰)NR²⁰R²⁰, aryl, aryloxy, arylthio, azido, carboxyl, —C(O)O—R²¹, —NR²⁰—C(O)O—R²¹, —O—C(O)O—R²¹, cyano, cycloalkyl, cycloalkyloxy, cycloalkylthio, —NR²⁰C(═NR²⁰)N(R²⁰)₂, halo, hydroxy, hydroxyamino, alkoxyamino, —NR²⁰NR²⁰R²⁰, heteroaryl, heteroaryloxy, heteroarylthio, heterocyclic, heterocyclyloxy, heterocyclylthio, nitro, spirocycloalkyl,
SO₃H, —OS(O)₂—R²¹, —S(O)₂—R²¹, —C(S)—R²¹, thiocyanate, thiol, and alkylthio; each R²⁰ is independently selected from the group consisting of hydrogen, alkyl, cycloalkyl, aryl, heteroaryl, and heterocycle, or two R²⁰ groups attached to a common atom are optionally joined together with the atom bound thereto to form a heterocycle; and each R²¹ is independently selected from the group consisting of alkyl, cycloalkyl, aryl, heteroaryl, and heterocycle.

“Cyano” refers to the group —CN.

“Cycloalkyl” refers to cyclic alkyl groups of from 3 to 10 or 3 to 8 carbon atoms having single or multiple cyclic rings including fused, bridged, and spiro ring systems. One or more of the rings can be aryl, heteroaryl, or heterocyclic provided that the point of attachment is through the non-aromatic, non-heterocyclic ring carbocyclic ring. Examples of suitable cycloalkyl groups include, for instance, adamantyl, cyclopropyl, cyclobutyl, cyclopentyl, and cyclooctyl. Other examples of cycloalkyl groups include bicycle[2,2,2,]octanyl, norbornyl, and spirobicyclo groups such as spiro[4.5]dec-8-yl.

“Substituted cycloalkyl” refers to a cycloalkyl group having from 1 to 5 or preferably 1 to 3 substituents selected from the group consisting of oxo, thione, alkyl, substituted alkyl, alkoxy, —C(O)—R²⁰, —NR²⁰C(O)R²⁰,

R²⁰—C(O)O—, —NR²⁰R²⁰, —C(O)NR²⁰R²⁰, —C(S)NR²⁰R²⁰, —NR²⁰C(O)NR²⁰R²⁰, —NR²⁰C(S)NR²⁰R²⁰, —O—C(O)NR²⁰R²⁰,
S(O)₂NR²⁰R²⁰, —O—S(O)₂NR²⁰R²⁰, —NR²⁰—S(O)₂NR²⁰R²⁰, —C(═NR²⁰)NR²⁰R²⁰, aryl, aryloxy, arylthio, azido, carboxyl, —C(O)O—R²¹, —NR²⁰—C(O)O—R²¹, —O—C(O)O—R²¹, cyano, cycloalkyl, cycloalkyloxy, cycloalkylthio, —NR²⁰C(═NR²⁰)N(R²⁰)₂, halo, hydroxy, hydroxyamino, alkoxyamino, —NR²⁰NR²⁰R²⁰, heteroaryl, heteroaryloxy, heteroarylthio, heterocyclic, heterocyclyloxy, heterocyclylthio, nitro, spirocycloalkyl,
SO₃H, —OS(O)₂—R²¹, —S(O)₂—R²¹, —C(S)—R²¹, thiocyanate, thiol, and alkylthio; each R²⁰ is independently selected from the group consisting of hydrogen, alkyl, cycloalkyl, aryl, heteroaryl, and heterocycle, or two R²⁰ groups attached to a common atom are optionally joined together with the atom bound thereto to form a heterocycle; and each R²¹ is independently selected from the group consisting of alkyl, cycloalkyl, aryl, heteroaryl, and heterocycle.

“Halo” or “halogen” refers to fluoro, chloro, bromo and iodo and preferably is fluoro or chloro.

“Haloalkyl” refers to alkyl groups substituted with 1 to 5, 1 to 3, or 1 to 2 halo groups, wherein alkyl and halo are as defined herein.

“Heteroaryl” refers to an aromatic group of from 5 to 14 ring atoms, including from 1 to 10 carbon atoms and 1 to 4 heteroatoms selected from the group consisting of oxygen, nitrogen and sulfur. In some embodiments, heteroaryl comprises 5, 6, or 7 ring atoms, including 1 to 4 heteroatoms. Such heteroaryl groups can have a single ring (e.g., pyridyl, pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, and/or sulfonyl moieties. Preferred heteroaryls include pyridinyl, pyrrolyl, indolyl, thiophenyl, and furanyl.

“Substituted heteroaryl” refers to heteroaryl groups that are substituted with from 1 to 5, preferably 1 to 3, or more preferably 1 to 2 substituents selected from the group consisting of the same group of substituents defined for substituted aryl.

“Heterocycle” or “heterocyclic” or “heterocycloalkyl” or “heterocyclyl” refers to a saturated or partially saturated, but not aromatic, group having from 3 to 14 ring atoms, including from 1 to 10 ring carbon atoms and from 1 to 4 ring heteroatoms selected from the group consisting of nitrogen, sulfur, or oxygen. In some embodiments, heteroaryl comprises 3, 4, 5, 6 or 7 ring atoms, including 1 to 4 heteroatoms. Heterocycle encompasses single ring or multiple condensed rings, including fused bridged and spiro ring systems. In fused ring systems, one or more the rings can be cycloalkyl, aryl, or heteroaryl provided that the point of attachment is through the non-aromatic heterocyclic ring. In one embodiment, the nitrogen and/or sulfur atom(s) of the heterocyclic group are optionally oxidized to provide for the N-oxide, sulfinyl, and/or sulfonyl moieties.

“Substituted heterocyclic” or “substituted heterocycloalkyl” or “substituted heterocyclyl” refers to heterocyclyl groups that are substituted with from 1 to 5 or preferably 1 to 3 of the same substituents as defined for substituted cycloalkyl.

“Ibogaine” refers to the compound:

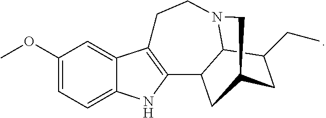

It should be understood that where “ibogaine” is mentioned herein, one more polymorphs of ibogaine can be utilized and are contemplated. Ibogaine is isolated from Tabernanth iboga, a shrub of West Africa. Ibogaine can also be synthesized using known methods. See, e.g., Biichi, et al. (1966), J. Am. Chem Society, 88(13), 3099-3109.

In some embodiments, the ibogaine or ibogaine derivative is represented by Formula I:

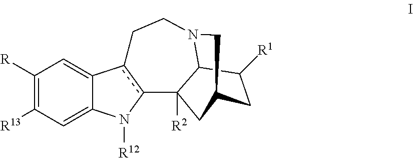

or a pharmaceutically acceptable salt and/or solvate thereof,
wherein
R is H, halo, C₁-C₃ alkyl, substituted C₁-C₃ alkyl, OR¹⁰, NH₂, NHR¹⁰, NR¹⁰R¹¹, NHC(O)R¹⁰, or NR¹⁰C(O)R¹¹;
R¹ is H, C₁-C₃ alkyl, substituted C₁-C₃ alkyl, C₁-C₃ alkoxy, CH₂—X—CH₃, or (CH₂)ₘR³;
R² is H, COOH, COOR⁴, (CH₂)˜OH, CH(OH)R⁵, CH₂OR⁵, C(O)NH₂, C(O)NHR⁵, C(O)NR⁵R⁶, C(O)NHNH₂, C(O)NHNHR⁵, C(O)NHNR⁵R⁶, C(O)NR⁵NH₂, C(O)NR⁵NHR⁶, C(O)NR⁵NR⁶R⁷, C(O)NHNH(C(O)R⁵), C(O)NHNR⁵(C(O)R⁶), C(O)NR⁵NH(C(O)R⁶), C(O)NR⁵NR⁶(C(O)R⁷), CN, or C(O)R⁵;
R³ is C₁-C₃ alkyl, benzyl, substituted C₁-C₃ alkyl, YH, YR⁸, YC(O)R⁸, C(O)YR⁸, C(O)NH₂, C(O)NHR⁸, C(O)NR⁸R⁹, NH₂, NHR⁸, NR⁸R⁹, NHC(O)R⁸, O(CH₂)ₚO(CH₂)_{q}O(CH₂)ᵣCH₃ or NR⁸C(O)R⁹;
R⁴ is C₁-C₆ alkyl or (CH₂CH₂O)˜CH₃;
R⁵, R⁶, R⁷, R⁸, R⁹, R¹⁰, and R¹¹ are independently alkyl or substituted alkyl;
R¹² is H, alkyl, or substituted alkyl;
R¹³ is H, OR¹⁰, alkyl, or substituted alkyl;
X is O or NH;
Y is O or S;
m is an integer selected from 0-8;
each of n, p and q is 1, 2 or 3; and
r is 0, 1 or 2.

In some embodiments, the ibogaine or ibogaine derivative is represented by Formula II:

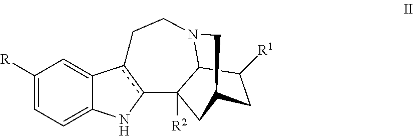

or a pharmaceutically acceptable salt and/or solvate thereof,
wherein
R is hydrogen or C₁-C₃ alkoxy,
R¹ is hydrogen, C₁-C₃ alkyl, C₁-C₃ alkoxy, (CH₂)ₘOC(O)alkyl, (CH₂)ₘOH, (CH₂)ₘOalkyl, (CH₂)ₘO(CH₂)ₚO(CH₂)_{q}O(CH₂)ᵣCH₃ or CH₂—Y—CH₃ where each of m, p and q is 1, 2 or 3; and r is 0, 1 or 2, Y is O or NH, and
R² is H, (CH₂)˜OH, COOH, or COOR⁴, where R⁴ is C₁-C₆ alkyl or (CH₂CH₂O)˜CH₃, where n is 1, 2, or 3.

In one embodiment, R is methoxy. In one embodiment, R¹ is ethyl. In one embodiment, R¹ is methoxy. In one embodiment, R¹ is CH₂—Y—CH₃ where Y is O. In one embodiment, R¹ is CH₂—Y—CH₃ where Y is NH. In one embodiment, R² is hydrogen. In one embodiment, In one embodiment, R² is COOR⁴ and R⁴ is methyl. In one embodiment, n=1. In a preferred embodiment, R, R¹ and R² are all not hydrogen. In one embodiment, when R is methoxy and R¹ is hydrogen, then R² is COOH or COOR⁴. In another embodiment, when R is methoxy and R¹ is hydrogen, then X is COOR⁴ where R⁴ is (CH₂CH₂O)CH₃.

In one embodiment, R¹² is hydrogen.

In one embodiment, R¹ is H. In one embodiment, R¹ is C₁-C₃ alkyl, such as ethyl. In one embodiment, R¹ is CH₂CH₂OH. In one embodiment, R¹ is CH₂CH₂OCH₃. In one embodiment, R¹ is CH₂CH₂OCH₂Ph. In one embodiment, R¹ is CH₂CH₂OC(O)alkyl. In one embodiment, R¹ is CH₂CH₂O(CH₂)ₚO(CH₂)_{q}O(CH₂)ᵣCH₃.

In one embodiment, R² is CH₂OH and CH(OH)R⁵. In one embodiment, R² is CH₂OR⁵. In one embodiment, R² is CO₂R⁵. In one embodiment, R² is C(O)NH₂, C(O)NHR⁵, or C(O)NR⁵R⁶. In one embodiment, R² is C(O)NHNH₂, C(O)NHNHR⁵, C(O)NR⁵NH₂, C(O)NHNR⁵R⁶, C(O)NH⁵NHR⁶, or C(O)NR⁵NR⁶R⁷. In one embodiment, R² is C(O)NHNH(C(O)R⁵), C(O)NHNR⁵(C(O)R⁶), C(O)NR⁵NH(C(O)R⁶), or C(O)NR⁵NR⁶(C(O)R⁷). In one embodiment, R² is C(O)R⁵.

In the various method, formulation and kit aspects and embodiments, in one embodiment a compound utilized herein is represented by, or ibogaine as used herein is replaced by, a compound Formula I:

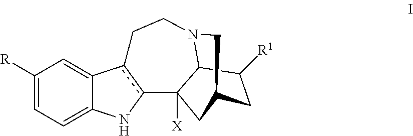

wherein
R is hydrogen or C₁-C₃-alkoxy,
R¹ is hydrogen, C₁-C₃-alkyl, C₁-C₃ alkoxy, or CH₂—Y—CH₃ where Y is O or NH, and
X is H, COOH, or COOR², where R² is C₁-C₆ alkyl or (CH₂CH₂O)˜CH₃, where n=1 to 3.

In another embodiment, ibogaine or a pharmaceutically acceptable salt and/or solvate thereof is utilized. In another embodiment, ibogaine or a pharmaceutically acceptable salt and/or solvate thereof is utilized. In another embodiment, the ibogaine, ibogaine derivative, is chosen from the group consisting of ibogaine, coronaridine, ibogamine, voacangine, 18-methoxycoronaridine, 2-methoxyethyl-18-methoxycoronaridinate, 18-methylaminocoronaridine or a pharmaceutically acceptable salt and/or solvate thereof.

In another embodiment, the compound utilized herein is chosen from the group consisting of ibogaine, coronaridine, ibogamine, voacangine, 18-methoxycoronaridine, 2-methoxyethyl-18-methoxycoronaridinate, 18-methylaminocoronaridine and a pharmaceutically acceptable salt and/or solvate.

In another embodiment, the compound utilized herein is selected from the group consisting of 16-hydroxymethyl-18-hydroxyibogaline, 16-hydroxymethyl-18-methoxyibogaline, 16-ethoxycarbonyl-18-hydroxyibogaline laurate, and 16-ethoxycarbonyl-18-hydroxyibogaline methoxyethoxymethyl ether and a pharmaceutically acceptable salt and/or solvate thereof.

In one embodiment, the ibogaine derivative is represented by Formula II:

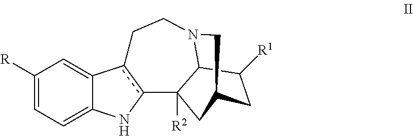

or a pharmaceutically acceptable salt and/or solvate thereof,
wherein
R is hydrogen or C₁-C₃ alkoxy;
R¹ is hydrogen, C₁-C₃ alkyl, C₁-C₃ alkoxy, (CH₂)ₘOC(O)alkyl, (CH₂)ₘOH, (CH₂)ₘOalkyl, (CH₂)ₘO(CH₂)ₚO(CH₂)_{q}O(CH₂)ᵣCH₃ or CH₂—Y—CH₃ where each of m, p and q is 1, 2 or 3; and r is 0, 1 or 2, Y is O or NH; and
R² is H, (CH₂)˜ OH, COOH, or COOR⁴, where R⁴ is C₁-C₆ alkyl or (CH₂CH₂O)˜ CH₃, where n is 1, 2, or 3.

In one embodiment, the ibogaine derivative is selected from the group consisting of coronaridine, ibogamine, voacangine, 18-methoxycoronaridine, 2-Methoxyethyl-18-methoxycoronaridinate, and 18-Methylaminocoronaridine.

In one embodiment, the ibogaine derivative is selected from the group consisting of 16-hydroxymethyl-18-hydroxyibogaline, 16-hydroxymethyl-18-methoxyibogaline, 16-ethoxycarbonyl-18-hydroxyibogaline laurate, and 16-ethoxycarbonyl-18-hydroxyibogaline methoxyethoxymethyl ether.

In one embodiment, the compound is of Formula IA:

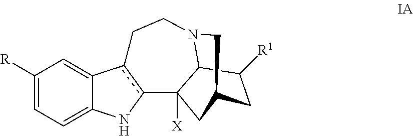

wherein
R is hydrogen or C₁-C₃-alkoxy,
R¹ is hydrogen, C₁-C₃-alkyl, C₁-C₃ alkoxy, or CH₂—Y—CH₃ where Y is O or NH, and
X is H, COOH, or COOR², where R² is C₁-C₆ alkyl or (CH₂CH₂O)˜CH₃, where n=1 to 3.

In another embodiment, the ibogaine derivative is represented by Formula II:

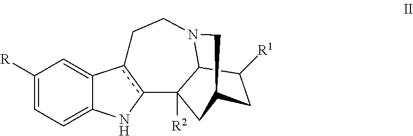

or a pharmaceutically acceptable salt and/or solvate thereof,
wherein
R is OCH₃;
R¹ is CH₂CH₃; and
   R² is COOR⁴, where R⁴ is (CH₂CH₂O)˜ CH₃, where n is 1.

When replacing ibogaine, the compounds of formula I, II, and subformulas thereof as utilized herein exclude ibogaine.

In a preferred embodiment, the compound utilized herein is:

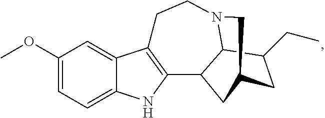

a pharmaceutically acceptable salt thereof, or a solvate of each thereof.

In some embodiments, the ibogaine or ibogaine derivative is selected from:

| Name | Structure |
|---|---|
| coronaridine | 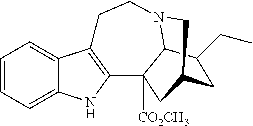 |
| 18-hydroxycoronaridine | 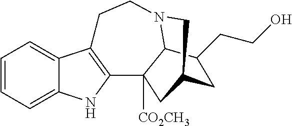 |
| 18-methoxycoronaridine | 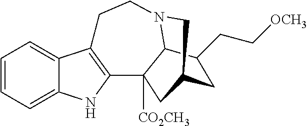 |
| 18-benzyloxycoronaridine | 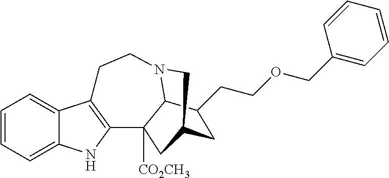 |
| 18-hydroxycoronaridine laurate | 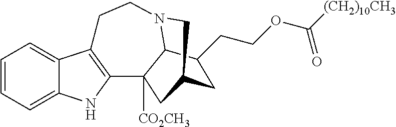 |
| 18-hydroxycoronaridine methoxyethoxymethyl ether | 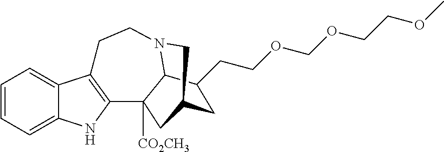 |
| 18-hydroxycoronaridine acetate | 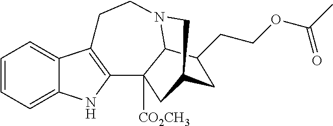 |
| voacangine | 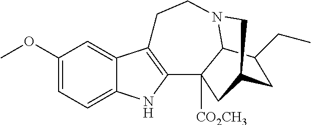 |
| 18-hydroxyvoacangine | 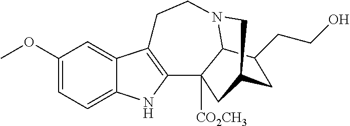 |
| 18-methoxyvoacangine | 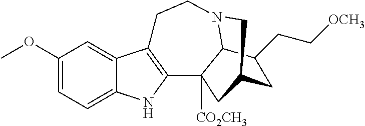 |
| 18-benzyloxyvoacangine | 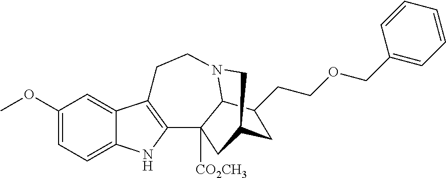 |
| 18-hydroxyvoacangine laurate | 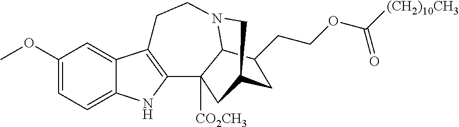 |
| 18-hydroxyvoacangine acetate | 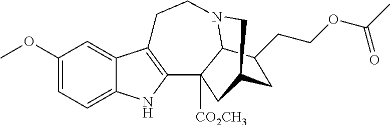 |
| 18-hydroxyvoacangine methoxyethoxymethyl ether | 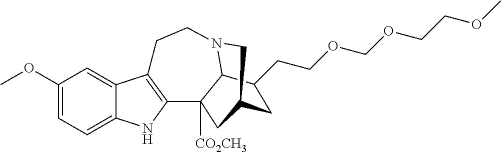 |
| conopharyngine | 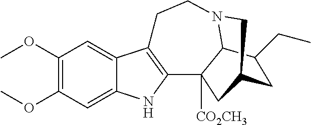 |
| 18-hydroxyconopharyngine | 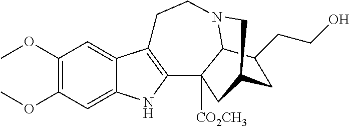 |
| 18-methoxyconopharyngine | 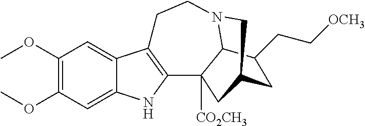 |
| 18-benzyloxyconopharyngine | 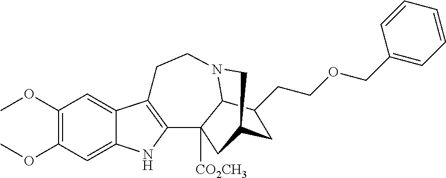 |
| 18-hydroxyconopharyngine laurate | 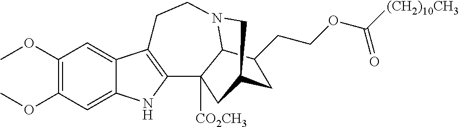 |
| 18-hydroxyconopharyngine acetate | 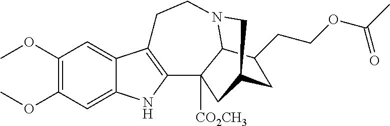 |
| 18-hydroxyconopharyngine methoxyethoxymethyl ether | 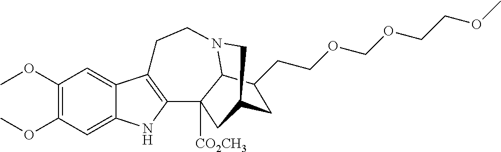 |
| ibogamine | 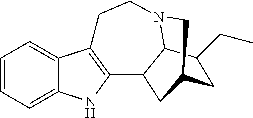 |
| 16-ethoxycarbonyl-18- hydroxyibogamine | 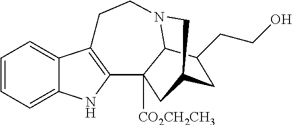 |
| 16-hydroxymethyl-18- hydroxyibogamine | 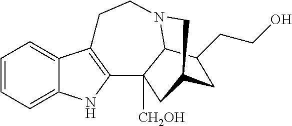 |
| 16-ethoxycarbonyl-18- methoxyibogamine | 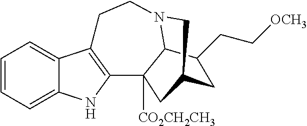 |
| 16-hydroxymethyl-18- methoxyibogamine | 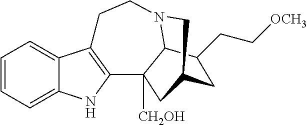 |
| 16-ethoxycarbonyl-18- benzyloxyibogamine | 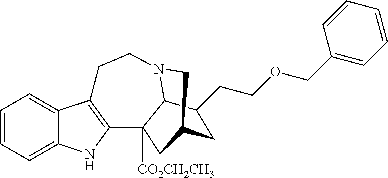 |
| 16-ethoxycarbonyl-18- hydroxyibogamine laurate | 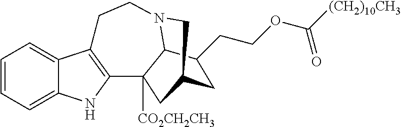 |
| 16-ethoxycarbonyl-18- hydroxyibogamine acetate | 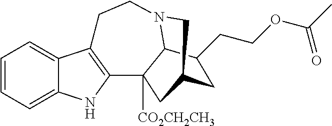 |
| 16-ethoxycarbonyl-18- hydroxyibogamine methoxyethoxymethyl ether | 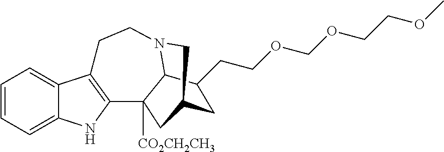 |
| ibogaine | 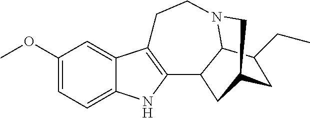 |
| 16-ethoxycarbonyl-18- hydroxyibogaine | 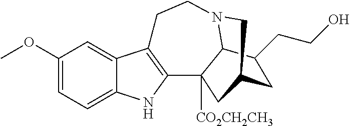 |
| 16-hydroxymethyl-18- hydroxyibogaine | 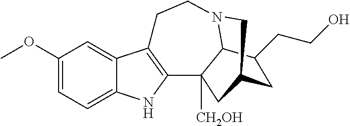 |
| 16-ethoxycarbonyl-18- methoxyibogaine | 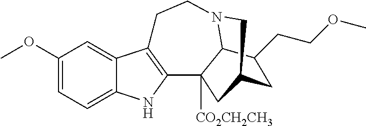 |
| 16-hydroxymethyl-18- methoxyibogaine | 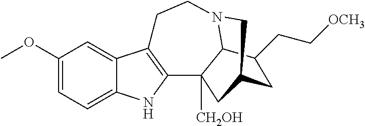 |
| 16-ethoxycarbonyl-18- benzyloxyibogaine | 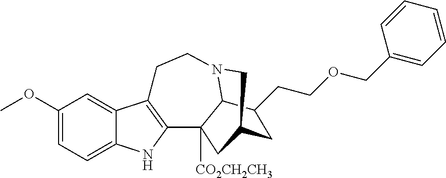 |
| 16-ethoxycarbonyl-18- hydroxyibogaine laurate | 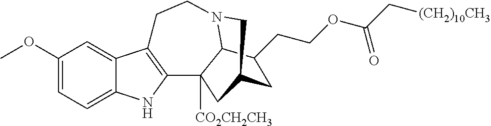 |
| 16-ethoxycarbonyl-18- hydroxyibogaine acetate | 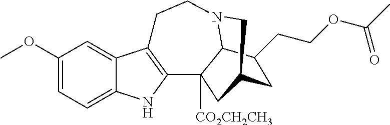 |
| 16-ethoxycarbonyl-18- hydroxyibogaine methoxyethoxymethyl ether | 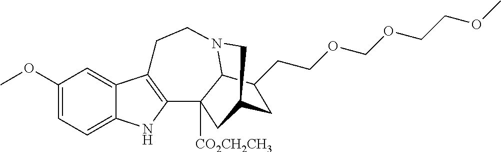 |
| ibogaline | 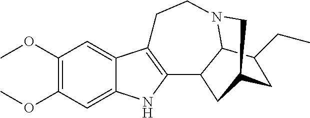 |
| 16-ethoxycarbonyl-18- hydroxyibogaline | 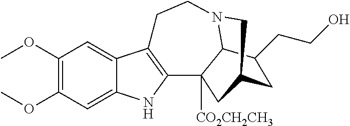 |
| 16-hydroxymethyl-18- hydroxyibogaline | 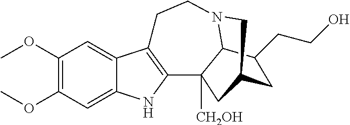 |
| 16-ethoxycarbonyl-18- methoxyibogaline | 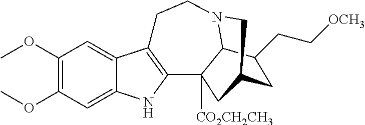 |
| 16-hydroxymethyl-18- methoxyibogaline | 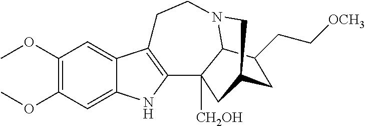 |
| 16-ethoxycarbonyl-18- benzyloxyibogaline | 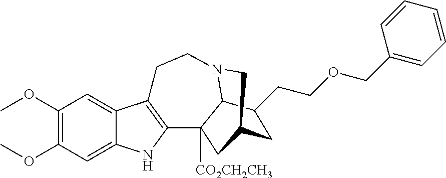 |
| 16-ethoxycarbonyl-18- hydroxyibogaline laurate | 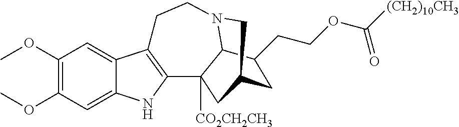 |
| 16-ethoxycarbonyl-18- hydroxyibogaline acetate | 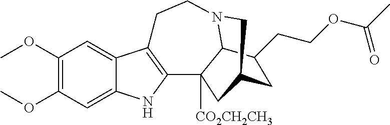 |
| 16-ethoxycarbonyl-18- hydroxyibogaline methoxyethoxymethyl ether | 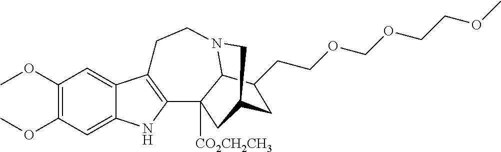 |

and pharmaceutically acceptable salts and/or solvates thereof.

In one embodiment, the ibogaine derivative is:

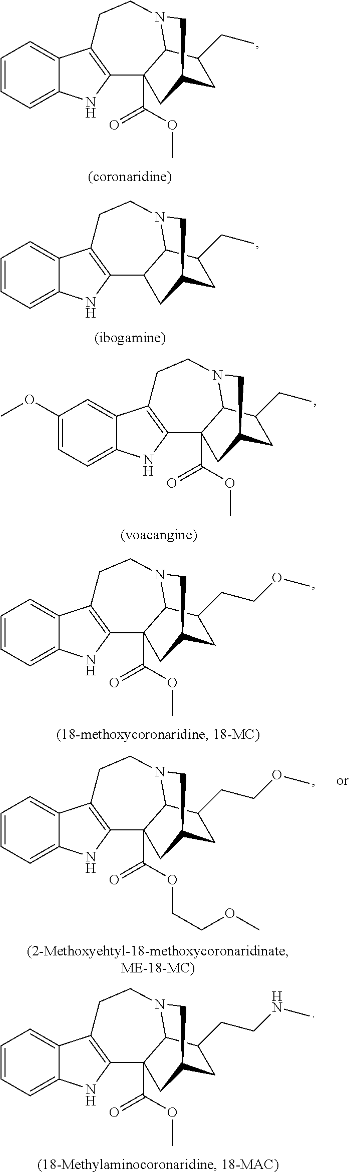

This invention is not limited to any particular chemical form of ibogaine or ibogaine derivative, and the drug may be given to patients either as a free base, solvate, or as a pharmaceutically acceptable acid addition salt. In the latter case, the hydrochloride salt is generally preferred, but other salts derived from organic or inorganic acids may also be used. Examples of such acids include, without limitation, those described below as “pharmaceutically acceptable salts” and the like.

“Pharmaceutically acceptable composition” refers to a composition that is suitable for administration to a human. Such compositions include various excipients, diluents, carriers, and such other inactive agents well known to the skilled artisan.

“Pharmaceutically acceptable salt” refers to pharmaceutically acceptable salts, including pharmaceutically acceptable partial salts, of a compound, which salts are derived from a variety of organic and inorganic counter ions well known in the art and include, by way of example only, hydrochloric acid, hydrobromic acid, phosphoric acid, sulfuric acid, methane sulfonic acid, phosphorous acid, nitric acid, perchloric acid, acetic acid, tartaric acid, lactic acid, succinic acid, citric acid, malic acid, maleic acid, aconitic acid, salicylic acid, thalic acid, embonic acid, enanthic acid, oxalic acid and the like, and when the molecule contains an acidic functionality, include, by way of example only, sodium, potassium, calcium, magnesium, ammonium, tetraalkylammonium, and the like.

A “pharmaceutically acceptable solvate or hydrate” of a compound of the invention means a solvate or hydrate complex that is pharmaceutically acceptable and that possesses the desired pharmacological activity of the parent compound, and includes, but is not limited to, complexes of a compound of the invention with one or more solvent or water molecules, or 1 to about 100, or 1 to about 10, or one to about 2, 3 or 4, solvent or water molecules.

As used herein the term solvate is taken to mean that a solid-form of a compound that crystallizes with one or more molecules of solvent trapped inside. A few examples of solvents that can be used to create solvates, such as pharmaceutically acceptable solvates, include, but are certainly not limited to, water, methanol, ethanol, isopropanol, butanol, C1-C6 alcohols in general (and optionally substituted), tetrahydrofuran, acetone, ethylene glycol, propylene glycol, acetic acid, formic acid, water, and solvent mixtures thereof. Other such biocompatible solvents which may aid in making a pharmaceutically acceptable solvate are well known in the art and applicable to the present invention. Additionally, various organic and inorganic acids and bases can be added or even used alone as the solvent to create a desired solvate. Such acids and bases are known in the art. When the solvent is water, the solvate can be referred to as a hydrate. Further, by being left in the atmosphere or recrystallized, the compounds of the present invention may absorb moisture, may include one or more molecules of water in the formed crystal, and thus become a hydrate. Even when such hydrates are formed, they are included in the term “solvate”. Solvate also is meant to include such compositions where another compound or complex co-crystallizes with the compound of interest.

“Therapeutically effective amount” or “therapeutic amount” refers to an amount of a drug or an agent that, when administered to a patient suffering from a condition, will have the intended therapeutic effect, e.g., alleviation, amelioration, palliation or elimination of one or more manifestations of the condition in the patient. The therapeutically effective amount will vary depending upon the patient and the condition being treated, the weight and age of the subject, the severity of the condition, the salt, solvate, or derivative of the active drug portion chosen, the particular composition or excipient chosen, the dosing regimen to be followed, timing of administration, the manner of administration and the like, all of which can be determined readily by one of ordinary skill in the art. The full therapeutic effect does not necessarily occur by administration of one dose, and may occur only after administration of a series of doses. Thus, a therapeutically effective amount may be administered in one or more administrations. For example, and without limitation, a therapeutically effective amount of ibogaine, in the context of modulating opioid analgesic tolerance, refers to an amount of ibogaine that resensitizes the patient to the opioid analgesic therapy.

A “therapeutic level” of a drug is an amount of ibogaine, ibogaine derivative, or pharmaceutical salt and/or solvate thereof that is sufficient to modulate tolerance to an opioid analgesic, but not high enough to pose any significant risk to the patient. Therapeutic levels of drugs can be determined by tests that measure the actual concentration of the compound in the blood of the patient. This concentration is referred to as the “serum concentration”.

Where the serum concentration of ibogaine is mentioned, it is to be understood that the term “ibogaine” encompasses any form of ibogaine, including derivatives thereof.

“Treatment,” “treating,” and “treat” are defined as acting upon a disease, disorder, or condition with an agent to reduce or ameliorate harmful or any other undesired effects of the disease, disorder, or condition and/or its symptoms. “Treatment,” as used herein, covers the treatment of a human patient, and includes: (a) reducing the risk of occurrence of the condition in a patient determined to be predisposed to the condition but not yet diagnosed as having the condition, (b) impeding the development of the condition, and/or (c) relieving the condition, i.e., causing regression of the condition and/or relieving one or more symptoms of the condition. “Treating” or “treatment of” a condition or patient refers to taking steps to obtain beneficial or desired results, including clinical results such as the reduction of symptoms. For purposes of this invention, beneficial or desired clinical results include, but are not limited to modulating, reducing, attenuating, relieving or reversing tolerance to an opioid analgesic compound.

“Nociceptive pain” refers to pain that is sensed by nociceptors, which are the nerves that sense and respond to parts of the body suffering from a damage. The nociceptors can signal tissue irritation, impending injury, or actual injury. When activated, they transmit pain signals (via the peripheral nerves as well as the spinal cord) to the brain. Nociceptive pain is typically well localized, constant, and often has an aching or throbbing quality. A subtype of nociceptive pain includes visceral pain and involves the internal organs. Visceral pain tends to be episodic and poorly localized. Nociceptive pain may be time limited; when the tissue damage heals, the pain typically resolves. However, nociceptive pain related to arthritis or cancer may not be time limited. Nociceptive pain tends to respond to treatment with opiate analgesics, such as, for example, buprenorphin, codeine, hydrocodone, oxycodone, morphine, and the like. Examples of nociceptive pain include, without limitation, pains from sprains, bone fractures, burns, bumps, bruises, inflammatory pain from an infection or arthritic disorder, pains from obstructions, cancer pain, and myofascial pain related to abnormal muscle stresses.

“Neuropathic pain” refers to chronic pain, often due to tissue injury. Neuropathic pain is generally caused by injury or damage to nerve fibers. It may include burning or coldness, “pins and needles” sensations, numbness and/or itching. It may be continuous and/or episodic. Neuropathic pain is difficult to treat, but opioids, including, without limitation, methadone, tramadol, tapentadol, oxycodone, methadone, morphine, levorphanol, and the like. Causes of neuropathic pain include, without limitation, alcoholism; amputation; back, leg, and hip problems; chemotherapy; diabetes; facial nerve problems; HIV/AIDS; multiple sclerosis; shingles; spine surgery; trigeminal neuralgia; fibromyalgia; and the like. In some cases, the cause of neuropathic pain may be unclear or unknown.

“Addictive” refers to a compound that, when administered to a mammal over a period of time, creates dependency in the mammal to that compound. The dependence can be physiological and/or psychological. A therapeutic effect of an addictive compound on a mammal may decrease with prolonged administration of the addictive compound, which is a non-limiting example of a physiological dependence. When administered to a mammal, an addictive compound may also create a craving in the mammal for more of it, which is a non-limiting example of a psychological dependence. Examples of addictive compounds include, without limitation, addictive opioids, and the like.

“Opioid” refers to a natural product or derivative thereof containing a basic nitrogen atom, typically as part of a cyclic ring structure and less commonly as an acyclic moiety, and synthetic derivatives thereof. Opioids include compounds extracted from poppy pods and their semi-synthetic counterparts which bind to the opiate receptors. Examples of opioids include, without limitation, buprenorphine, codeine, heroine, hydrocodone, oxycodone, morphine, thebaine, and their derivatives, which will be well known to the skilled artisan.

“Analgesic” and “analgesic agent” refer to a compound that is capable of inhibiting and/or reducing pain in mammals. Pain may be inhibited and/or reduced in the mammal by the binding of the opioid analgesic agent to the mu receptor. When analgesia is effected through the mu receptor, the analgesic agent is referred to as a mu receptor agonist. Certain analgesic agents are capable of inhibiting nociceptive and/or neuropathic pain including, by way of example, morphine, codeine, hydromorphone, oxycodone, hydrocodone, buprenorphin, and the like.

As used herein, the term “patient” refers to mammals and includes humans and non-human mammals.

As used herein, the term “QT interval” refers to the measure of the time between the start of the Q wave and the end of the T wave in the electrical cycle of the heart. Prolongation of the QT interval refers to an increase in the QT interval.

The term “tolerance” as used herein refers to the psychological and/or physiologic process wherein the patient adjusts to the frequent presence of a substance such that a higher dose of the substance is required to achieve the same effect. Tolerance may develop at different times for different effects of the same drug (e.g., analgesic effect versus side effects). The mechanisms of tolerance are not entirely understood, but they may include receptor down-regulation or desensitization, inhibitory pathway up-regulation, increased metabolism, and/or changes in receptor processing (e.g., phosphorylation).

A “pharmaceutically acceptable solvate or hydrate” of a compound of the invention means a solvate or hydrate complex that is pharmaceutically acceptable and that possesses the desired pharmacological activity of the parent compound, and includes, but is not limited to, complexes of a compound of the invention with one or more solvent or water molecules, or 1 to about 100, or 1 to about 10, or one to about 2, 3 or 4, solvent or water molecules.

### II. Methods of the Invention

As will be apparent to the skilled artisan upon reading this disclosure, the present invention provides a method for modulating tolerance to opioids in a patient undergoing opioid analgesic therapy, comprising administering to the patient a dosage of ibogaine, ibogaine derivative, or pharmaceutically acceptable salt and/or solvate thereof.

In one aspect of this invention, patient is being treated with an addictive opioid analgesic to relieve the patient's pain. The pain may be of any type and from any source. In one embodiment, the patient is treated for acute pain. In one embodiment, the patient is treated for chronic pain. In one embodiment, the patient is treated for nociceptive pain. In one embodiment, the patient is treated for neuropathic pain. In some embodiments, the pain is caused by surgery, diabetes, trigeminal neuralgia, fibromyalgia, cancer, central pain syndrome, tissue damage, physical injury, and the like. In some embodiments, the source of the pain is unknown or unclear.

In one aspect, this invention relates to a method for modulating tolerance to an opioid analgesic in a patient undergoing opioid analgesic therapy, the method comprising interrupting or administering concurrently with said opioid analgesic an amount of ibogaine, ibogaine derivative, or pharmaceutically acceptable salt and/or solvate thereof that provides an average serum concentration of about 50 ng/mL to about 850 ng/mL, said concentration being sufficient resensitize the patient to the opioid as an analgesic while maintaining a QT interval of less than about 500 ms during said treatment.

In one aspect, this invention relates to a method for modulating tolerance to an opioid analgesic in a patient undergoing opioid analgesic therapy, the method comprising interrupting or administering concurrently with said opioid analgesic an amount of ibogaine, ibogaine derivative, or pharmaceutically acceptable salt and/or solvate thereof that provides an average serum concentration of about 60 ng/mL to about 400 ng/mL, said concentration being sufficient to resensitize the patient to the opioid as an analgesic while maintaining a QT interval of less than about 500 ms during said treatment. In some embodiments, the concentration is sufficient to resensitize the patient to the opioid as an analgesic while maintaining a QT interval of less than about 470 ms during treatment. Preferably, the concentration is sufficient to resensitize the patient to the opioid as an analgesic while maintaining a QT interval of less than about 450 ms during treatment. In one embodiment, the concentration is sufficient to resensitize the patient to the opioid as an analgesic while maintaining a QT interval of less than about 420 ms during treatment.

In one embodiment, the average serum concentration of ibogaine is from about 50 ng/mL to about 800 ng/mL or about 60 ng/mL to about 800 ng/mL. In one embodiment, the average serum concentration of ibogaine is from about 50 ng/mL to about 700 ng/mL or about 60 ng/mL to about 700 ng/mL. In one embodiment, the average serum concentration of ibogaine is from about 50 ng/mL to about 600 ng/mL, or about 60 ng/mL to about 600 ng/mL. In a preferred embodiment, the average serum concentration of ibogaine is from about 50 ng/mL to about 500 ng/mL, or about 60 ng/mL to about 500 ng/mL. In one embodiment, the average serum concentration of ibogaine is from about 50 ng/mL to about 400 ng/mL, or about 60 ng/mL to about 400 ng/mL. In one embodiment, the average serum concentration of ibogaine is from about 50 ng/mL to about 300 ng/mL, or about 60 ng/mL to about 300 ng/mL. In one embodiment, the average serum concentration of ibogaine is from about 50 ng/mL to about 200 ng/mL, or about 60 ng/mL to about 200 ng/mL. In one embodiment, the average serum concentration of ibogaine is from about 50 ng/mL to about 100 ng/mL, or about 60 ng/mL to about 100 ng/mL. The ranges include both extremes as well as any subranges therebetween.

In one embodiment, the dosage or aggregate dosage of ibogaine, ibogaine derivative, or pharmaceutically acceptable salt and/or solvate thereof is from greater than about 1 mg/kg to about 8 mg/kg body weight per day. The aggregate dosage is the combined dosage, for example the total amount of ibogaine, ibogaine derivative, or pharmaceutically acceptable salt and/or solvate thereof administered over a 24-hour period where smaller amounts are administered more than once per day. In one embodiment, the dosage or aggregate dosage of ibogaine, ibogaine derivative, or salt and/or solvate thereof is from about 1.3 mg/kg to about 7 mg/kg body weight. In one embodiment, the dosage or aggregate dosage of ibogaine, ibogaine derivative, or salt and/or solvate thereof is from about 1.3 mg/kg to about 6 mg/kg body weight. In one embodiment, the dosage or aggregate dosage of ibogaine, ibogaine derivative, or salt and/or solvate thereof is from about 1.3 mg/kg to about 5 mg/kg body weight. In a preferred embodiment, the dosage or aggregate dosage of ibogaine, ibogaine derivative, or salt and/or solvate thereof is from about 1.3 mg/kg to about 4 mg/kg body weight. In one embodiment, the dosage or aggregate dosage of ibogaine, ibogaine derivative, or salt and/or solvate thereof is from about 1.3 mg/kg to about 3 mg/kg body weight. In one embodiment, the dosage or aggregate dosage of ibogaine, ibogaine derivative, or salt and/or solvate thereof is from about 1.3 mg/kg to about 2 mg/kg body weight. In one embodiment, the dosage or aggregate dosage of ibogaine, ibogaine derivative, or salt and/or solvate thereof is from about 1.5 mg/kg to about 3 mg/kg body weight. In one embodiment, the dosage or aggregate dosage of ibogaine, ibogaine derivative, or salt and/or solvate thereof is from about 1.7 mg/kg to about 3 mg/kg body weight. In one embodiment, the dosage or aggregate dosage of ibogaine, ibogaine derivative, or salt and/or solvate thereof is from about 2 mg/kg to about 4 mg/kg body weight. In one embodiment, the dosage or aggregate dosage of ibogaine, ibogaine derivative, or salt and/or solvate thereof is from about 2 mg/kg to about 3 mg/kg body weight. In one embodiment, the dosage or aggregate dosage of ibogaine, ibogaine derivative, or salt and/or solvate thereof is about 2 mg/kg body weight. The ranges include both extremes as well as any subranges there between.

In one embodiment, the dosage or aggregate dosage of ibogaine, ibogaine derivative, or salt and/or solvate thereof is about 8 mg/kg body weight per day. In one embodiment, the dosage or aggregate dosage of ibogaine, ibogaine derivative, or salt and/or solvate thereof is about 7 mg/kg body weight per day. In one embodiment, the dosage or aggregate dosage of ibogaine, ibogaine derivative, or salt and/or solvate thereof is about 6 mg/kg body weight per day. In one embodiment, the dosage or aggregate dosage of ibogaine, ibogaine derivative, or salt and/or solvate thereof is about 5 mg/kg body weight per day. In one embodiment, the dosage or aggregate dosage of ibogaine, ibogaine derivative, or salt and/or solvate thereof is about 4 mg/kg body weight per day. In one embodiment, the dosage or aggregate dosage of ibogaine, ibogaine derivative, or salt and/or solvate thereof is about 3 mg/kg body weight per day. In one embodiment, the dosage or aggregate dosage of ibogaine, ibogaine derivative, or salt and/or solvate thereof is about 2 mg/kg body weight per day. In one embodiment, the dosage or aggregate dosage of ibogaine, ibogaine derivative, or salt and/or solvate thereof is about 1.7 mg/kg body weight per day. In one embodiment, the dosage or aggregate dosage of ibogaine, ibogaine derivative, or salt and/or solvate thereof is about 1.5 mg/kg body weight per day. In one embodiment, the dosage or aggregate dosage of ibogaine, ibogaine derivative, or salt and/or solvate thereof is about 1.3 mg/kg body weight per day. In one embodiment, the dosage or aggregate dosage of ibogaine, ibogaine derivative, or salt and/or solvate thereof is about 1 mg/kg body weight per day.

In one embodiment, ibogaine is administered at an amount by weight that is twice that administered for noribogaine for treating a same or similar condition. For example, and without limitation, an administration of a dose 80 mg ibogaine approximates a dose of 40 mg noribogaine.

In one embodiment, the QT interval is not prolonged more than about 50 ms. In one embodiment, the QT interval is not prolonged more than about 40 ms. In one embodiment, the QT interval is not prolonged more than about 30 ms. In one embodiment, the QT interval is not prolonged more than about 20 ms. In one embodiment, the QT interval is not prolonged more than about 10 ms.

In some embodiments, the patient is administered periodically, such as once, twice, three times, four times or five times daily with ibogaine, ibogaine derivative, or a pharmaceutically acceptable salt and/or solvate thereof. In some embodiments, the administration is once daily, or once every second day, once every third day, three times a week, twice a week, or once a week. The dosage and frequency of the administration depends on the route of administration, dosage, age and body weight of the patient, condition of the patient, opioid analgesic to which tolerance is being modulated, length of time of analgesic treatment, and the like, without limitation. Determination of dosage and frequency suitable for the present technology can be readily made a qualified clinician.

Ibogaine, ibogaine derivative, or a pharmaceutically acceptable salt and/or solvate thereof, suitable for administration in accordance with the methods provide herein, can be suitable for a variety of delivery modes including, without limitation, oral and transdermal delivery. Compositions suitable for internal, pulmonary, rectal, nasal, vaginal, lingual, intravenous, intra-arterial, intramuscular, intraperitoneal, intracutaneous and subcutaneous routes may also be used. Possible dosage forms include tablets, capsules, pills, powders, aerosols, suppositories, parenterals, and oral liquids, including suspensions, solutions and emulsions. Sustained release dosage forms may also be used. All dosage forms may be prepared using methods that are standard in the art (see e.g., Remington's Pharmaceutical Sciences, 16th ed., A. Oslo editor, Easton Pa. 1980).

In a preferred embodiment, ibogaine, ibogaine derivative, or a pharmaceutically acceptable salt and/or solvate thereof is administered orally, which may conveniently be provided in tablet, caplet, sublingual, liquid or capsule form. In certain embodiments, the ibogaine is provided as ibogaine HCl, with dosages reported as the amount of free base ibogaine. In some embodiments, the ibogaine HCl is provided in hard gelatin capsules containing only ibogaine HCl with no excipients.

The patient may be receiving any addictive opioid analgesic for the treatment of pain. In a preferred embodiment, the opioid analgesic is selected from the group consisting of fentanyl, hydrocodone, hydromorphone, morphine, oxycodone, buprenorphine, codeine, heroin, thebaine, buprenorphine, methadone, meperidine, tramadol, tapentadol, levorphanol, sufentanil, pentazocine, oxymorphone, and derivatives of each thereof.

### Patient Pre-Screening and Monitoring

Pre-screening of patients before treatment with ibogaine, ibogaine derivative, or pharmaceutically acceptable salt and/or solvate thereof and/or monitoring of patients during ibogaine treatment may be required to ensure that QT interval is not prolonged beyond a certain value. For example, QT interval greater than 500 ms can be considered dangerous for individual patients. Pre-screening and/or monitoring may be necessary at high levels of ibogaine treatment.

In one embodiment, a patient receiving a therapeutic dose of ibogaine, ibogaine derivative, or pharmaceutically acceptable salt and/or solvate thereof is monitored in a clinical setting. Monitoring may be necessary to ensure the QT interval is not prolonged to an unacceptable degree. A “clinical setting” refers to an inpatient setting (e.g., inpatient clinic, hospital, rehabilitation facility) or an outpatient setting with frequent, regular monitoring (e.g., outpatient clinic that is visited daily to receive dose and monitoring). Monitoring includes monitoring of QT interval. Methods for monitoring of QT interval are well-known in the art, for example by ECG.

In one embodiment, a patient administered ibogaine, ibogaine derivative, or pharmaceutically acceptable salt and/or solvate thereof is not monitored in a clinical setting. In one embodiment, a patient administered ibogaine, ibogaine derivative, or pharmaceutically acceptable salt and/or solvate thereof is monitored periodically, for example daily, weekly, monthly, or occasionally.

In one aspect, this invention relates to a method for modulating tolerance to an opioid analgesic in a patient undergoing opioid analgesic therapy, comprising selecting a patient who is prescreened to evaluate the patient's expected tolerance for prolongation of QT interval, and administering to the patient a dosage of ibogaine, ibogaine derivative, or pharmaceutically acceptable salt and/or solvate thereof that provides an average serum concentration of about 50 ng/mL to about 850 ng/mL, said concentration being sufficient to resensitize the patient to the opioid as an analgesic while maintaining a QT interval of less than 500 ms during said treatment. In some embodiments, the concentration is sufficient to resensitize the patient to the opioid as an analgesic while maintaining a QT interval of less than about 470 ms during treatment. Preferably, the concentration is sufficient to resensitize the patient to the opioid as an analgesic while maintaining a QT interval of less than about 450 ms during treatment. In one embodiment, the concentration is sufficient to resensitize the patient to the opioid as an analgesic while maintaining a QT interval of less than about 420 ms during treatment.

In one embodiment, prescreening of the patient comprises ascertaining that ibogaine treatment will not result in a QT interval over about 500 ms. In one embodiment, prescreening of the patient comprises ascertaining that ibogaine treatment will not result in a QT interval over about 470 ms. In one embodiment, prescreening comprises ascertaining that ibogaine treatment will not result in a QT interval over about 450 ms. In one embodiment, prescreening comprises ascertaining that ibogaine treatment will not result in a QT interval over about 420 ms. In one embodiment, prescreening comprises determining the patient's pre-treatment QT interval.

As it relates to pre-screening or pre-selection of patients, patients may be selected based on any criteria as determined by the skilled clinician. Such criteria may include, by way of non-limiting example, pre-treatment QT interval, pre-existing cardiac conditions, risk of cardiac conditions, age, sex, general health, and the like. The following are examples of selection criteria for disallowing ibogaine treatment or restricting dose of ibogaine administered to the patient: high QT interval before treatment (e.g., such that there is a risk of the patient's QT interval exceeding 500 ms during treatment); congenital long QT syndrome; bradycardia; hypokalemia or hypomagnesemia; recent acute myocardial infarction; uncompensated heart failure; and taking other drugs that increase QT interval. In some embodiments, the methods can include selecting and/or administering/providing ibogaine to a patient that lacks one more of such criteria.

In one embodiment, this invention relates to pre-screening a patient to determine if the patient is at risk for prolongation of the QT interval beyond a safe level. In one embodiment, a patient at risk for prolongation of the QT interval beyond a safe level is not administered ibogaine. In one embodiment, a patient at risk for prolongation of the QT interval beyond a safe level is administered ibogaine, ibogaine derivative, or pharmaceutically acceptable salt and/or solvate thereof at a limited dosage. In one embodiment, a limited dosage is a dosage of ibogaine, ibogaine derivative, or pharmaceutically acceptable salt and/or solvate thereof that is not expected to result in an unacceptable prolongation of the patient's QT interval.

In one embodiment, this invention relates to monitoring a patient who is administered a therapeutic dose of ibogaine, ibogaine derivative, or salt and/or solvate thereof. In one embodiment, the dose of ibogaine is reduced if the patient has one or more adverse side effects. In one embodiment, the ibogaine treatment is discontinued if the patient has one or more adverse side effects. In one embodiment, the adverse side effect is a QT interval that is prolonged beyond a safe level. The determination of a safe level of prolongation is within the skill of a qualified clinician.

### Kit of Parts

One aspect of this invention is directed to a kit of parts for the modulation of tolerance to an opioid analgesic, wherein the kit comprises a composition comprising ibogaine, ibogaine derivative, or salt and/or solvate thereof and a means for administering the composition to a patient in need thereof. The means for administration to a patient can include, for example, any one or combination of ibogaine, or a ibogaine derivative, or a pharmaceutically acceptable salt and/or solvate thereof, a transdermal patch, a syringe, a needle, an IV bag comprising the composition, a vial comprising the composition, an inhaler comprising the composition, etc. In one embodiment, the kit of parts further comprises instructions for dosing and/or administration of the composition.

In some aspects, the invention is directed to a kit of parts for administration of ibogaine, ibogaine derivative, pharmaceutically acceptable salt and/or solvate thereof, the kit comprising multiple delivery vehicles, wherein each delivery vehicle contains a discrete amount of ibogaine, ibogaine derivative, pharmaceutically acceptable salt and/or solvate thereof and further wherein each delivery vehicle is identified by the amount of ibogaine, ibogaine derivative, pharmaceutically acceptable salt and/or solvate thereof provided therein; and optionally further comprising a dosing treatment schedule in a readable medium. In some embodiments, the dosing treatment schedule includes the amount of ibogaine, ibogaine derivative, pharmaceutically acceptable salt and/or solvate thereof required to achieve each average serum level is provided. In some embodiments, the kit of parts includes a dosing treatment schedule that provides an attending clinician the ability to select a dosing regimen of ibogaine based on criteria such as, without limitation, the sex of the patient, mass of the patient, and the serum level that the clinician desires to achieve. In some embodiments, the dosing treatment schedule further provides information corresponding to the volume of blood in a patient based upon weight (or mass) and sex of the patient. In an embodiment, the storage medium can include an accompanying pamphlet or similar written information that accompanies the unit dose form in the kit. In an embodiment, the storage medium can include electronic, optical, or other data storage, such as a non-volatile memory, for example, to store a digitally-encoded machine-readable representation of such information.

The term “delivery vehicle” as used herein refers to any formulation that can be used for administration of ibogaine, ibogaine derivative, pharmaceutically acceptable salt and/or solvate thereof to a patient. Non-limiting, exemplary delivery vehicles include caplets, pills, capsules, tablets, powder, liquid, or any other form by which the drug can be administered. Delivery vehicles may be intended for administration by oral, inhaled, injected, or any other means.

The term “readable medium” as used herein refers to a representation of data that can be read, for example, by a human or by a machine. Non-limiting examples of human-readable formats include pamphlets, inserts, or other written forms. Non-limiting examples of machine-readable formats include any mechanism that provides (i.e., stores and/or transmits) information in a form readable by a machine (e.g., a computer, tablet, and/or smartphone). For example, a machine-readable medium includes read-only memory (ROM); random access memory (RAM); magnetic disk storage media; optical storage media; and flash memory devices. In one embodiment, the machine-readable medium is a CD-ROM. In one embodiment, the machine-readable medium is a USB drive. In one embodiment, the machine-readable medium is a Quick Response Code (QR Code) or other matrix barcode.

In some aspects, the machine-readable medium comprises software that contains information regarding dosing schedules for the unit dose form of ibogaine and optionally other drug information. In some embodiments, the software may be interactive, such that the attending clinician or other medical professional can enter patient information. In a non-limiting example, the medical professional may enter the weight and sex of the patient to be treated, and the software program provides a recommended dosing regimen based on the information entered. The amount and timing of ibogaine recommended to be delivered will be within the dosages that result in the serum concentrations as provided herein.

In some embodiments, the kit of parts comprises multiple delivery vehicles in a variety of dosing options. For example, the kit of parts may comprise pills or tablets in multiple dosages, such as 240 mg, 120 mg, 90 mg, 60 mg, 30 mg, 20 mg, and/or 10 mg of ibogaine per pill. Each pill is labeled such that the medical professional and/or patient can easily distinguish different dosages. Labeling may be based on printing or embossing on the pill, shape of the pill, color of pill, the location of the pill in a separate, labeled compartment within the kit, and/or any other distinguishing features of the pill. In some embodiments, all of the delivery vehicles within a kit are intended for one patient. In some embodiments, the delivery vehicles within a kit are intended for multiple patients.

One aspect of this invention is directed to a kit of parts for the modulation of tolerance to an opioid analgesic in a patient undergoing opioid analgesic therapy, wherein the kit comprises a unit dose form of ibogaine, ibogaine derivative, salt and/or solvate thereof. The unit dose form provides a patient with an average serum level of ibogaine of from about 50 ng/mL to about 800 ng/mL or about 60 ng/mL to about 800 ng/mL. In one embodiment, the unit dose form provides a patient with an average serum level of ibogaine of from about 50 ng/mL to about 400 ng/mL or about 60 ng/mL to about 400 ng/mL.

In some embodiments, the unit dose form comprises one or multiple dosages to be administered periodically, such as once, twice, three time, four times or five time daily with ibogaine, ibogaine derivative, or pharmaceutically acceptable salt and/or solvate thereof. In some embodiments, the administration is once daily, or once every second day, once every third day, three times a week, twice a week, or once a week. The dosage and frequency of the administration depends on criteria including the route of administration, content of composition, age and body weight of the patient, condition of the patient, sex of the patient, without limitation, as well as by the opioid analgesic employed. Determination of the unit dose form providing a dosage and frequency suitable for a given patient can readily be made by a qualified clinician.

These dose ranges may be achieved by transdermal, oral, or parenteral administration of ibogaine, ibogaine derivative, or a pharmaceutically acceptable salt and/or solvate thereof in unit dose form. Such unit dose form may conveniently be provided in transdermal patch, tablet, caplet, liquid or capsule form. In certain embodiments, the ibogaine is provided as ibogaine HCl, with dosages reported as the amount of free base ibogaine. In some embodiments, the ibogaine HCl is provided in hard gelatin capsules containing only ibogaine HCl with no excipients. In some embodiments, ibogaine is provided in saline for intravenous administration.

### Formulations

This invention further relates to pharmaceutically acceptable formulations comprising a unit dose of ibogaine, ibogaine derivative, or pharmaceutically acceptable salt and/or solvate thereof, wherein the amount of ibogaine, ibogaine derivative, or pharmaceutically acceptable salt and/or solvate thereof is sufficient to provide an average serum concentration of about 50 ng/mL to about 850 ng/mL when administered to a patient. In a preferred embodiment, the amount of ibogaine, ibogaine derivative, or pharmaceutically acceptable salt and/or solvate thereof is sufficient to provide an average serum concentration of about 50 ng/mL to about 400 ng/mL when administered to a patient.

In some embodiments, the unit dose of ibogaine, ibogaine derivative, or pharmaceutically acceptable salt and/or solvate thereof is administered in one or more dosings.

In one embodiment, the amount of ibogaine, ibogaine derivative, or pharmaceutically acceptable salt and/or solvate thereof is sufficient to provide an average serum concentration of about 50 ng/mL to about 800 ng/mL or about 60 ng/mL to about 800 ng/mL. In one embodiment, the amount of compound is sufficient to provide an average serum concentration of about 50 ng/mL to about 700 ng/mL or about 60 ng/mL to about 700 ng/mL. In one embodiment, the amount of compound is sufficient to provide an average serum concentration of about 50 ng/mL to about 600 ng/mL, or about 60 ng/mL to about 600 ng/mL. In a preferred embodiment, the amount of compound is sufficient to provide an average serum concentration of about 50 ng/mL to about 500 ng/mL, or about 60 ng/mL to about 500 ng/mL. In one embodiment, the amount of compound is sufficient to provide an average serum concentration of about 50 ng/mL to about 400 ng/mL, or about 60 ng/mL to about 400 ng/mL. In one embodiment, the amount of compound is sufficient to provide an average serum concentration of about 50 ng/mL to about 300 ng/mL, or about 60 ng/mL to about 300 ng/mL. In one embodiment, the amount of compound is sufficient to provide an average serum concentration of about 50 ng/mL to about 200 ng/mL, or about 60 ng/mL to about 200 ng/mL. In one embodiment, the amount of compound is sufficient to provide an average serum concentration of about 50 ng/mL to about 100 ng/mL, or about 60 ng/mL to about 100 ng/mL. The ranges include both extremes as well as any subranges between.

In some embodiments, the formulation is designed for periodic administration, such as once, twice, three time, four times or five time daily with ibogaine, ibogaine derivative, or a pharmaceutically acceptable salt and/or solvate thereof. In some embodiments, the administration is once daily, or once every second day, once every third day, three times a week, twice a week, or once a week. The dosage and frequency of the administration depends on the route of administration, content of composition, age and body weight of the patient, condition of the patient, without limitation. Determination of dosage and frequency suitable for the present technology can be readily made a qualified clinician.

In some embodiments, the formulation designed for administration in accordance with the methods provide herein can be suitable for a variety of delivery modes including, without limitation, oral and transdermal delivery. Formulations suitable for internal, pulmonary, rectal, nasal, vaginal, lingual, intravenous, intra-arterial, intramuscular, intraperitoneal, intracutaneous and subcutaneous routes may also be used. Possible formulations include tablets, capsules, pills, powders, aerosols, suppositories, parenterals, and oral liquids, including suspensions, solutions and emulsions. Sustained release dosage forms may also be used. All formulations may be prepared using methods that are standard in the art (see e.g., Remington's Pharmaceutical Sciences, 16th ed., A. Oslo editor, Easton Pa. 1980).

In a preferred embodiment, the formulation is designed for oral administration, which may conveniently be provided in tablet, caplet, sublingual, liquid or capsule form. In certain embodiments, the ibogaine is provided as ibogaine HCl, with dosages reported as the amount of free base ibogaine. In some embodiments, the ibogaine HCl is provided in hard gelatin capsules containing only ibogaine HCl with no excipients.

### EXAMPLES

The following Examples are intended to further illustrate certain embodiments of the disclosure and are not intended to limit its scope.

### Example 1. Effect of Ibogaine, Ibogaine Derivative, or a Pharmaceutically Acceptable Salt and/or Solvate Thereof on QT Interval in Humans

The effect of ibogaine, ibogaine derivative, or a pharmaceutically acceptable salt and/or solvate thereof on QT interval is evaluated in substance-dependent participants in a randomized, placebo-controlled, double-blind trial. Patients are administered 60 mg or 120 mg of the compound and QT interval is measured.

### Example 2. Efficacy of Ibogaine to Modulate Opioid Tolerance in Humans

A female patient, age 59, undergoing opioid analgesic therapy for chronic back pain, is treated with ibogaine hydrochloride at a dose of about 2 mg/kg concurrently with the opioid. The amount of opioid required to treat her back pain after ibogaine treatment is measured.

1. A method for modulating tolerance to an opioid analgesic in a patient undergoing opioid analgesic therapy, the method comprising interrupting or administering concurrently with said opioid analgesic therapy an amount of an ibogaine derivative or pharmaceutically acceptable salt and/or solvate thereof that provides an average serum concentration of about 50 ng/mL to about 500 ng/mL, said concentration being sufficient to re-sensitize the patient to the opioid as an analgesic while maintaining a QT interval of less than about 500 ms during said treatment.

2. The method of claim 1, wherein the ibogaine derivative; or pharmaceutically acceptable salt and/or solvate thereof is administered as a single dose or multiple doses.

3. The method of claim 1, further comprising interrupting the dosage of the analgesic.

4. The method of claim 1, further comprising administering the ibogaine derivative; or pharmaceutically acceptable salt and/or solvate thereof concurrently with the analgesic.

5. The method of claim 4, wherein during concurrent administration, the dose of opioid analgesic is reduced.

6. The method of claim 1, wherein the dose or aggregate dosage of the ibogaine derivative; or pharmaceutically acceptable salt and/or solvate thereof is from about 1.3 mg/kg to about 4 mg/kg per day.

7. The method of claim 1, wherein the aggregate dosage of the ibogaine derivative; or pharmaceutically acceptable salt and/or solvate thereof is from about 1.5 mg/kg to about 3 mg/kg per day.

8. The method of claim 1, wherein the aggregate dosage of the ibogaine derivative; or pharmaceutically acceptable salt and/or solvate thereof is from about 2 mg/kg to about 4 mg/kg per day.

9. The method of claim 1, wherein the aggregate dosage of the ibogaine derivative; or pharmaceutically acceptable salt and/or solvate thereof is from about 2 mg/kg to about 3 mg/kg per day.

10. The method of claim 1, wherein the aggregate dosage of the ibogaine derivative; or pharmaceutically acceptable salt and/or solvate thereof is about 2 mg/kg per day.

11. The method of claim 1, wherein the dosage of the ibogaine derivative; or pharmaceutically acceptable salt and/or solvate thereof provides an average serum concentration of about 50 ng/mL to about 200 ng/mL.

12. The method of claim 1, wherein the QT interval is less than about 470 ms.

13. The method of claim 1, wherein the QT interval is less than about 450 ms.

14. The method of claim 1, further comprising selecting a patient who is prescreened to evaluate tolerance for prolongation of QT interval.

15. The method of claim 14, wherein the prescreening step comprises ascertaining that ibogaine derivative treatment will not result in a QT interval greater than about 500 ms.

16. The method of claim 15, wherein the prescreening step comprises ascertaining that ibogaine derivative treatment will not result in a QT interval greater than about 470 ms.

17. The method of claim 16, wherein the prescreening step comprises ascertaining that ibogaine derivative treatment will not result in a QT interval greater than about 450 ms.

18. The method of claim 1, wherein the opioid analgesic is selected from the group consisting of fentanyl, hydrocodone, hydromorphone, morphine, oxycodone, buprenorphine, codeine, thebaine, buprenorphine, methadone, meperidine, tramadol, tapentadol, levorphanol, sufentanil, pentazocine, oxymorphone.

19. The method of claim 18, wherein the opioid analgesic is morphine.

20. The method of claim 1, wherein the ibogaine derivative is represented by Formula I: 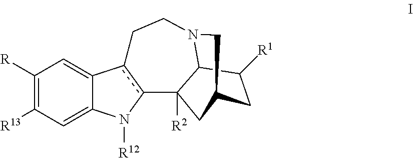
or a pharmaceutically acceptable salt and/or solvate thereof, wherein R is H, halo, C₁-C₃ alkyl, substituted C₁-C₃ alkyl, OR¹⁰, NH₂, NHR¹⁰, NR¹⁰R¹¹, NHC(O)R¹⁰, or NR¹⁰C(O)R¹¹; R¹ is H, C₁-C₃ alkyl, substituted C₁-C₃ alkyl, C₁-C₃ alkoxy, CH₂—X—CH₃, or (CH₂)ₘR³; R² is H, COOH, COOR⁴, (CH₂)ₙOH, CH(OH)R⁵, CH₂OR⁵, C(O)NH₂, C(O)NHR⁵, C(O)NR⁵R⁶, C(O)NHNH₂, C(O)NHNHR⁵, C(O)NHNR⁵R⁶, C(O)NR⁵NH₂, C(O)NR⁵NHR⁶, C(O)NR⁵NR⁶R⁷, C(O)NHNH(C(O)R⁵), C(O)NHNR⁵(C(O)R⁶), C(O)NR⁵NH(C(O)R⁶), C(O)NR⁵NR⁶(C(O)R⁷), CN, or C(O)R⁵; R³ is C₁-C₃ alkyl, benzyl, substituted C₁-C₃ alkyl, YH, YR⁸, YC(O)R⁸, C(O)YR⁸, C(O)NH₂, C(O)NHR⁸, C(O)NR⁸R⁹, NH₂, NHR⁸, NR⁸R⁹, NHC(O)R⁸, O(CH₂)ₚO(CH₂)_{q}O(CH₂)ᵣCH₃ or NR⁸C(O)R⁹; R⁴ is C₁-C₆ alkyl or (CH₂CH₂O)ₙCH₃; R⁵, R⁶, R⁷, R⁸, R⁹, R¹⁰, and R¹¹ are independently alkyl or substituted alkyl; R¹² is H, alkyl, or substituted alkyl; R¹³ is H, OR¹⁰, alkyl, or substituted alkyl; X is O or NH; Y is O or S; m is an integer selected from 0-8; each of n, p and q is 1, 2 or 3; and r is 0, 1 or 2, provided that when R¹ is ethyl and R², R¹² and R¹³ are H then R is not OMe.

21. The method of claim 1, wherein the ibogaine derivative is represented by Formula II: 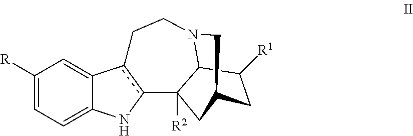
or a pharmaceutically acceptable salt and/or solvate thereof, wherein R is hydrogen or C₁-C₃ alkoxy, R¹ is hydrogen, C₁-C₃ alkyl, C₁-C₃ alkoxy, (CH₂)ₘOC(O)alkyl, (CH₂)ₘOH, (CH₂)ₘOalkyl, (CH₂)ₘO(CH₂)ₚO(CH₂)_{q}O(CH₂)ᵣCH₃ or CH₂—Y—CH₃ where each of m, p and q is 1, 2 or 3; and r is 0, 1 or 2, Y is O or NH, and R² is H, (CH₂)ₙOH, COOH, or COOR⁴, where R⁴ is C₁-C₆ alkyl or (CH₂CH₂O)ₙCH₃, where n is 1, 2, or 3, provided that when R¹ is ethyl and R² is H then R is not OMe.

22. The method of claim 1, wherein the ibogaine derivative is represented by the Formula IA: 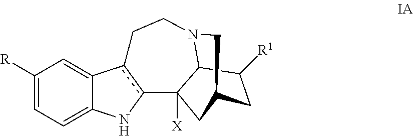
wherein R is hydrogen or C₁-C₃-alkoxy, R¹ is hydrogen, C₁-C₃-alkyl, C₁-C₃ alkoxy, or CH₂—Y—CH₃ where Y is O or NH, and X is H, COOH, or COOR², where R² is C₁-C₆ alkyl or (CH₂CH₂O)ₙCH₃, where n is 1 to 3, provided that when R¹ is ethyl and X is H then R is not OMe.

23. The method of claim 1, wherein the ibogaine derivative is selected from the group consisting of coronaridine, ibogamine, voacangine, 18-methoxycoronaridine, 2-methoxyethyl-18-methoxycoronaridinate, 18-methylaminocoronaridine or a pharmaceutically acceptable salt and/or solvate thereof.

24. The method of claim 1, wherein the ibogaine derivative is selected from the group consisting of 16-hydroxymethyl-18-hydroxyibogaline, 16-hydroxymethyl-18-methoxyibogaline, 16-ethoxycarbonyl-18-hydroxyibogaline laurate, and 16-ethoxycarbonyl-18-hydroxyibogaline methoxyethoxymethyl ether and a pharmaceutically acceptable salt and/or solvate thereof.

25. The method of claim 23, wherein the ibogaine derivative is 18-methoxycoronaridine or a pharmaceutically acceptable salt and/or solvate thereof.
